(19) United States
(12) Patent Application Publication
(10) Pub. No.: US 2013/0129802 A1
(43) Pub. Date: May 23, 2013

(54) GLASS FLAKES AND THEIR MANUFACTURING METHOD

(71) Applicant: Simon James Brigham, Perth (AU)

(72) Inventor: Simon James Brigham, Perth (AU)

(21) Appl. No.: 13/808,590

(22) PCT Filed: Jul. 6, 2011
(86) PCT No.: PCT/GB11/01020

§ 371 Date: Feb. 11, 2013

(30) Foreign Application Priority Data
Jul. 7, 2010 (GB) 1011443.7

(51) Int. Cl. A61K 8/02; A61Q 3/02; A61Q 19/04; A61Q 1/10; A61Q 1/06; A61Q 1/00

(52) U.S. Cl. CPC A61K 8/0254; A61Q 1/06; A61Q 1/00; A61Q 19/04; A61Q 1/10; A61Q 3/02
USPC 424/401; 428/402; 428/406; 65/21.2

(57) ABSTRACT

A method for manufacturing glass flakes by a rotary method includes the steps of using slow fed impact millers and multiple passings through opposed jet mills to achieve a low mean thickness deviation. The glass flakes, which may be used in cosmetic formulations, have a thickness of less than 500 nm, a particle size of 5-100 microns and a mean thickness deviation of less than 75 nm.

### TECHNICAL FIELD

The present invention relates ultra thin glass flakes and their use as a filler in cosmetic formulations.

### BACKGROUND ART

Glass flakes have been used for a substantial time as a filler to improve material properties and it has been found that glass flakes give a significant benefit both as a functional and decorative pigment in cosmetic formulations.

In cosmetic formulations fillers are employed to enhance properties such as skin feel, adhesion, and appearance on the skin and also to bulk out the formulation. They may also have functional benefits. Common fillers that are employed include minerals such as mica, talc, kaolin, sericite, calcium carbonate and silica. Polymers may also be employed such as nylon powder, polymethylmethacrylate, melamine resins. Other fillers that are employed include BiOCl, barium sulphate, calcium sulphate and boron nitride.

### STATEMENTS OF THE INVENTION

According to the present invention, there is provided glass flakes having a thickness of <500 nm and a particle size of 5-100 microns and mean thickness deviation of <75 nm.

Glass flakes of the invention have uniform shape in contrast to traditional fillers. They are light stable, mechanically stable and do not discolour. Furthermore their morphology can be controlled to enhance their effects.

EP089240 describes a rotary process for producing glass flake. EP089240 has a practical limitation of producing product down to 275 nm. Furthermore the deviation and spread of thicknesses is too broad for use in cosmetics. Flake of the present invention is not only a thinner flake but also one that has a more tightly controlled mean thickness deviation, and greater flake uniformity. Glass flakes relating to the invention, in contrast to glass flakes produced by the standard rotary process, have an improved appearance and an improved skin feel. Furthermore, due to the low bulk density, there is an economical benefit.

The glass flakes of the invention are preferably made using a process such as that described in EP2203394. The process involves feeding molten glass into a cup mounted for rotation about a longitudinal axis, rotating the cup about said axis whereby a film of molten glass is caused to emanate radially from the rim of the cup. Insulating means extends at least partially around the cup and/or the cup is heated while rotating.

In order to obtain a thickness deviation of less that 75 nm, the process of EP2203394 is modified by subjecting the flake produced by the process to the action of slow fed impact millers such as hammer millers under reduced vacuum and then subjecting the flake to multiple passing through opposed jet mills.

### DETAILED DESCRIPTION OF THE INVENTION

In contrast to the prior art, the glass flakes according to the invention exhibit a better behaviour as a filler in cosmetic formulations owing to the higher control of platelet morphology in particular the control over deviation. Prior art describes the use of glass flakes at given thicknesses for use in cosmetic applications, typically material produced according to EP089240 or (NSG patent citation) has a deviation of +/−2 microns and at best +/−200 nm. Product with such deviation in the average thickness has an uneven appearance and skin feel and is not desirable.

Suitable glasses include silicate glasses such as borosilicate glass, soda-lime glass, aluminosilicate, C, A, S, R, E, ECR, low alkali glasses. Glass flakes may be amorphous, exhibit a degree of crystallinity or phase separation depending on the particular application of use. Such glasses may be produced from mineral oxides or pre-melted marbles. For the purpose of the invention it is preferred to produce melt from the mineral oxides to control the exact composition and, for the purpose of personal care cosmetic formulations, the heavy metal content.

The glass flakes to be used in the present embodiment are preferably substantially free from so-called heavy metals such as lead and arsenic as components of the composition.

The glass flakes have a thickness of <1 micron with a mean deviation of 75 nm, in particular 25-500 nm with a mean deviation of 75 nm, and preferably 50-275 nm with a mean deviation of 25-50 nm. The average particle size is 5-100 microns, preferably 5-40 microns.

The glass flakes have an aspect ratio of 1 to 100, preferably 1 to 175, and more preferably 1 to 200.

Glass flakes are produced from melt using known processes such as that of the bubble process and the rotary process. For the purpose of the invention the rotary process described in EP0289240 is preferred. However the process as described does not allow for glass flake to be produced with a reasonable mean thickness of below 275 nm. Furthermore, the product produced has a large thickness deviation. At best such glass flake has a ratio of 1 or less using the formula ΔD=(D90-D10)/D50 where Dx is the particle size where x% of the particles have a thickness greater than D and (100−x)% of the particles have a thickness less than D. For example glass flake produced by the rotary method of EP0289240 produces a product that has the following characteristics: D10 35; D50 110; D90 140 giving a ratio ΔD of 0.5. The particle size can however be adjusted by secondary processing including impact mills, opposed jet mills, sonic vibratory sieves or classification.

Glass flakes according to the invention display different physical characteristics compared to flakes that have an average thickness above 500 nm, thinner the flake the more flexible and malleable. Therefore the conventional particle reducing methods used for glass flakes which rely on inter particle impact or direct impact are inadequate to achieve the desired particle size for use in the present invention where a ratio of 1.0-2.5 is preferred. Furthermore, a low deviation particle bulk density as a result in the low deviation in flake thickness causes further issues in obtaining narrow fractions and reducing the ratio of fines or oversized particles that are detrimental to the overall effect achieved.

To overcome these problems the flake is processed using modified slow fed impact millers such as a hammer millers under reduced vacuum. The product then undergoes multiple passings of opposed jet mills to achieve the desired particle size diameters. The invention allows for the production of glass flakes with a mean thicknesses from 25-500 nm, with mean thickness deviations of +/−25-50 nm.

Examples of particle size distribution of glass flake of the invention are as follows:

### D10 7.5 D50 20 D90 57

ΔD=(D90-D10)/D50

(57-7.5)/20=2.47 Examble 1A

### D10 5 D50 12 D90 25

ΔD=(D90-D10)/D50

(25−5)/12=1.66 Example 2B

The concentration of the glass flakes in a formulation, such as a cosmetic formulation, is generally between 0.1 and 95% by weight, preferably between 0.1 and 50% by weight and more preferably between 1.0 and 10% by weight, based on the total solids content of the system. It is generally dependent on the specific application and can be up to 95% by weight in the case of loose powders.

The glass flakes according to the invention are simple and easy to handle since they can be incorporated into a formulation very easily. The glass flakes can be incorporated into the application system alone or in the form of a mixture with further cosmetic assistants, carriers and active compounds by simple stirring-in. Complex dispersal of the glass flakes is unnecessary.

The glass flakes according to the invention can be combined in the formulations with any type of cosmetic raw materials and assistants and active compounds. These include, inter alia, water, alcohols, polyols, polar and non-polar oils, fats, waxes, film formers, polymers, copolymers, surfactants, free-radical scavengers, antioxidants, such as, for example, vitamin C or vitamin E, stabilisers, odour enhancers, silicone oils, emulsifiers, fragrances, solvents, such as, for example, ethanol, ethyl acetate or butyl acetate, preservatives and assistants which generally determine the application properties, such as, for example, thickeners and rheological additives, such as, for example, bentonites, hectorites, silicon dioxides, Ca silicates, gelatine, high-molecular-weight carbohydrates and/or surface-active assistants, etc.

Suitable active compounds are, for example, insect repellents, inorganic UV filters, such as, for example, TiO₂, UV A/B protection filters (for example OMC, BP-3 , 4-MBC), including in encapsulated form, anti-ageing active compounds, vitamins and derivatives thereof (for example vitamin A, C, E, etc.), self-tanning agents (for example DHA, erythrulose, inter alia) and further cosmetic active compounds, such as, for example, bisabolol, LPO, VTA, ectoine, emblica, allantoin, bioflavonoids and derivatives thereof.

Organic UV filters are generally incorporated into cosmetic formulations in an amount of 0.5 to 10% by weight, preferably 1 to 8% by weight, and inorganic UV filters are incorporated in an amount of 0.1 to 30% by weight. The compositions according to the invention may, in addition, comprise further conventional skin-protecting or skin-care active compounds. These may in principle be all active compounds known to the person skilled in the art. Particularly preferred active compounds are pyrimidinecarboxylic acids and/or aryl oximes.

The glass flakes may be modified by way of compositional changes to add functional or decorative effects. The glass flakes according to the invention may advantageously be used as a mixture with colourants in cosmetics, in particular, effect pigments, such as, for example, pearlescent pigments, interference pigments, goniochromatic pigments, BiOCl flakes, multilayered pigments, metal pigments, organic dyes, organic coloured pigments and other pigments, such as, for example, transparent and opaque white, coloured and black pigments, and also with flake-form iron oxides, holographic pigments, LCPs (liquid crystal polymers) and conventional transparent, coloured and black lustre pigments based on metal-oxide-coated mica flakes and SiO.sub.2 flakes, etc. The glass flakes according to the invention can be mixed with commercially available (effect) pigments in any ratio. The glass flake:pigment weight ratio can be 1:99 to 99:1, depending on the colour intensity. In the case of coloured textures, the pigment proportion is higher than in the case of less-coloured textures.

Glass flakes of the invention may be coloured as described in PCT/GB2010/00376. Colouration, in particular black, brown, blue, golden, green and red, may be achieved by adding inorganic compounds. Specific functionality such as photochromic, thermochromic, UV absorption, IR absorption may also be achieved.

There are no restrictions regarding the type effect including substrate type and coating including metal oxides and pure metals or combinations thereof.

The coloured pigments, such as, for example, anthraquinone pigments, quinacridone pigments, diketopyrrolopyrrole pigments, phthalocyanine pigments, azo pigments, isoindoline pigments. The needle-shaped pigments are preferably BiOCl, coloured glass fibres, .alpha.-FeOOH, organic colour pigments, such as, for example, azo pigments, beta.-phthalocyanine Cl Blue 15.3, Cromophtal Yellow 8GN (Ciba-Geigy), Irgalith Blue PD56 (Ciba-Geigy), azomethine/copper complex Cl Yellow 129, Irgazine Yellow 5GT (Ciba-Geigy).

Suitable organic coloured pigments and dyes are of natural or synthetic origin, such as, for example, chromium oxide and ultramarine.

The glass flakes, which are preferably used as fillers in cosmetic formulations, may of course also be mixed or employed with other known fillers. Fillers which may be mentioned are, for example, synthetic organic polymers, polymethylmethacrylate, methyl methacrylate cross polymer, natural and synthetic mica, nylon powder, pure or filled melamine resins, talc, SiO.sub.2, glass powder, glass beads, kaolin, oxides or hydroxides of aluminium, magnesium, calcium, zinc, BiOCl, barium sulfate, calcium sulfate, calcium carbonate, magnesium carbonate, basic alkaline-earth metal carbonates, such as, for example, calcium carbonate or magnesium carbonate, carbon, and physical or chemical combinations of these substances.

Nanoscale dielectrics may also be incorporated in order to improve the skin feel further. Examples of additions of this type are Al.sub.2O.sub.3, SiO.sub.2, ZnO or TiO.sub.2, which are usually added to the formulation in amounts of 0.01-15% by weight.

The formulations comprising the glass flakes according to the invention can belong to the lipophilic, hydrophilic or hydrophobic type. In the case of heterogeneous formulations having discrete aqueous and non-aqueous phases, the glass flakes according to the invention may be present in each case in only one of the two phases or alternatively distributed over both phases.

The pH values of the formulations can be between 1 and 14, preferably between 2 and 11 and particularly preferably between 5 and 8.

Application forms of the cosmetic formulations which may be mentioned are, for example, solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower preparations. Besides the glass flakes according to the invention, any desired customary carriers, assistants and, if desired, further active compounds may be added to the composition.

Ointments, pastes, creams and gels may comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary carriers, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary carriers, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary carriers, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary carriers, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary carriers, such as synthetic oils, such as, for example, fatty acid esters, fatty alcohols, silicone oils, natural oils, such as vegetable oils and oily plant extracts, paraffin oils, lanolin oils, or mixtures of these substances.

The cosmetic compositions may exist in various forms. Thus, they can be, for example, a solution, a water-free composition, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Further embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

Solid sticks consist of natural or synthetic waxes and oils, fatty alcohols, fatty acids, fatty acid esters, lanolin and other fatty substances.

Cosmetic oils are preferably mineral oil, hydrogenated polyisobutene, synthetic squalane or squalane prepared from natural products, cosmetic esters or ethers, which may be branched or unbranched, saturated or unsaturated, vegetable oils or mixtures thereof.

The cosmetic composition may also be used to protect the hair against photochemical damage in order to prevent colour changes, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling or treating the hair, in the form of a lotion or gel for brushing or blow-waving, in the form of a hair lacquer, permanent waving composition, colorant or bleach for the hair. The composition having light-protection properties may comprise assistants, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The glass flakes according to the invention can be used as fillers, for example in lipsticks, lip gloss, rouge, eyeliner, eye shadow, (volume) mascara, nail varnishes, day creams, night creams, body lotions, cleansing milk, body powder, hair gels, hair masks, hair rinses, hair shampoos, shower gels, shower oils, bath oils, sunscreen, pre-sun and after-sun preparations, tanning lotions, tanning sprays, make-ups, lotions, soaps, bath salts, toothpastes, face masks, compact powders, loose powders and gels, etc. Products of this type are produced in a manner as is known to the person skilled in the art in this area.

The present invention furthermore relates to cosmetic formulations comprising the glass substrates according to the invention.

The invention thus also relates to the use of the glass flakes according to the invention as filler, in particular in care and decorative cosmetics, and in paints, coatings, automobile paints, powder coatings, printing inks, security printing inks, plastics, paper, in paper coatings, in pigment pastes with water, organic and/or aqueous solvents, for the preparation of pigment compositions and dry preparations, such as, for example, granules. The glass flakes according to the invention are preferably employed as functional filler since the glass flakes may improve the application properties, the skin feel, the pay-off and the compressibility of powders. Furthermore, they may reduce the fatty, sticky aspect of the formulation, increase the richness of emulsions, and influence the viscosity properties.

The surface of a glass flake is inherently hydrophilic, and has less affinity for oils to be added during manufacturing a cosmetic. Thus, when the glass flake needs to have an affinity for oils, the surface thereof may be coated with a hydrophobizing agent. In this case, the entire surface of the glass flake may be coated with the hydrophobizing agent, or a part of the surface may be coated.

Examples of the hydrophobizing agents that can be used for this coating treatment include, as components usually to be used for surface treatment of cosmetic pigments: silicone compounds such as methylhydrogenpolysiloxane, reactive alkylpolysiloxane, high-viscosity silicone oil, and silicone resin; surfactants such as an anion activator and a cation activator; polymer compounds such as a silane coupling agent, a titania coupling agent, nylon, polymethylmethacrylate, polyethylene, fluororesin, and polyamino acid; hydrogenated lecithin; acylating collagen; metallic soap; lipophilic wax; and polyhydric alcohol ester. These hydrophobizing agents may be used in combination.

Examples of coating treatment methods using hydrophobizing agents include a liquid spraying method, and a liquid dipping method, and others. The adhesion amount of the hydrophobizing agent is normally 0.01 to 5 mass % per mass of glass flakes. As a result of the coating treatment, a cosmetic with a better feel can be obtained.

Glass flakes according to the invention may have a further coating to improve the compatibility with the medium it is added to, these coatings include known surface modifiers such as silanes and stearates. By example Triethoxycaprylyl silane for use in cosmetic formulations; 3-Aminopropyltriethoxy silane for use in polymer and coatings systems.

### EXAMPLES

The following examples further illustrate the invention.

### Example 1

A lipstick containing glass flake according to the invention gives a brilliant shimmer effect and may be prepared in the normal manner by combining the ingredients in separate phases and conditions and subsequently combining to produce the end article. The formulation is as follows:

| | % wt |
|---|---|
| Beeswax | 8 |
| Carnauba Wax | 6 |
| Candelilla Wax | 8 |
| Ozokerite | 5 |
| Polyisobutene | 29 |
| Castor oil | 20 to 45 |
| Glass flake coated with metal oxide or | 3.5 |
| pure metal | |
| Red 7 Lake | 2.25 |
| Tocopherol acetate | 0.05 |
| BHT | 0.03 |
| Peppermint | 0.3 |

### Example 2

A face foundation containing glass flakes according to the invention gives an exceptionally smooth skin feel and colour to complement skin tones.

The formulation may be prepared in the normal manner by combining the ingredients in a number of phases and conditions and subsequently combining to produce the end article. The formulation is as follows:

| | % wt |
|---|---|
| Mineral oil | 6.0 |
| Stearic acid | 1.9 |
| Glyceryl stearate | 1.8 |
| Lanolin alcohol | 3 |
| Cetearyl isononanoate | 1 |
| Isostearic acid | 0.5 |
| Cellulose gum | 0.3 |
| Xanthan gum | 0.3 |
| Propylene glycol | 4.7 |
| Triethanolamine | 0.9 |
| Deionized water | As required |
| Glass flake with inherent colour | 0.2 |
| Titanium dioxide | 2 |
| Glass flake coated with metal oxide | 7.8 |
| Phenoxyethanol, methylparaben, | 0.7 |
| ethylparaben, propylparaben, | |
| butylparaben, isobutylparaben | |

### Example 3

A self tanning lotion, containing glass flakes of the invention, for both decorative and functional effects may be prepared in the normal manner by combining the ingredients in a number of phases and conditions and subsequently combining to produce the end article. The formulation is as follows:

| | % wt |
|---|---|
| Gylceryl stearate, steareth-25, ceteth-20, | 8 |
| stearyl alcohol | |
| Stearyl alcohol | 1 |
| Cetearyl ethylhexanoate | 5.5 |
| Caprylic/capric triglyceride | 5.5 |
| Tearoxy dimethicone | 0.5 |
| Dimethicone | 0.5 |
| Glassflake coated with metal oxide or | 1.2 |
| metal | |
| Glycerin | 1.8 |
| Water (aqua) | 63 |
| Erythrulose | 2.8 |
| Sodium metabisulfite | 0.2 |
| Deionised Water (aqua) | 9.2 |
| Caprylyl glycol, phenoxyethanol, | |
| hexylene glycol | 0.8 |

### Example 4

A pressed powder eye shadow formulation contains glass flakes of the invention and is a replacement for talc to give improved appearance and skin feel.

The formulation may be prepared in the normal manner by combining the ingredients in a number of phases and conditions and subsequently combining to produce the end article. The formulation is as follows:

| | % wt |
|---|---|
| Glassflake | 50.00 |
| Mica | 10.00 |
| Magnesium Myristate | 5.50 |
| Silica | 1.50 |
| Glassflake with metal oxide | 15.00 |
| Preservatives | as required |
| Octyl Palmitate | 6.00 |
| Isostearyl Neopentanoate | 2.00 |
| Glassflake with inherent colour and metal oxide coatings | 10.00 |

### Example 5

A nail varnish containing glass flake according to the invention gives a brilliant shimmer effect. The formulation is as follows:

| INGREDIENTS | % wt |
|---|---|
| Suspending Lacquer SLF-2 (Butyl Acetate, Toluene, Nitrocellulose, | 93 |
| Tosylamide/Formaldehyde Resin, Isopropyl Alcohol, Dibutyl | |
| Phthalate, Ethyl Acetate, Camphor, n-Butyl Alcohol, | |
| Glass flakes (and) Quaterinum-18 Hectorite) | |
| Glassflake coated with a metal oxide or metal oxide or combination | 3 |
| thereof | |
| Lacquer 127P | 4 |

1-19. (canceled)

20. Glass flakes having a thickness of less than 500 nm, a particle size of 5-100 microns and a mean thickness deviation of less than 75 nm.

21. The glass flakes according to claim 20, wherein the glass flakes have a thickness of less than 275 nm and the mean thickness deviation is less than 50 nm.

22. The glass flakes according to claim 20, wherein the glass flakes have a thickness of less than 250 nm and the mean thickness deviation is less than 25 nm.

23. The glass flakes according to claim 20, wherein the glass flakes have a thickness of less than 200 nm and the mean thickness deviation is less than 75 nm.

24. The glass flakes according to claim 20, wherein the glass flakes are mass-colored with at least one inorganic compound.

25. The glass flakes according to claim 20, wherein the glass flakes are photochromic, thermochromic, IR absorption, UVA absorption or UVB absorption.

26. The glass flakes according to claim 20, wherein the glass flakes have an aspect ratio of 1:200.

27. The glass flakes according to claim 20, wherein the glass flakes include a member selected from the group consisting of borsilicate glass, soda-lime glass, aluminosilicate, C, A, S, R, E, ECR, a low-alkali glass and a combination thereof.

28. The glass flakes according claim 20, wherein the glass flakes comprise a ceramic material.

29. The glass flakes according to claim 20, wherein the glass flakes are substantially free of heavy metals.

30. The glass flakes according to claim 20, wherein the glass flakes are at least one of being amorphous, crystalline, exhibit a degree of crystallinity, or have a phase-separated depending composition.

31. A cosmetic composition, comprising:
a cosmetic formulation comprising glass flakes having a thickness of less than 500 nm, a particle size of 5-100 microns and a mean thickness deviation of less than 75 nm.

32. The cosmetic composition according to claim 31, wherein said cosmetic formulation is a lipophilic, hydrophilic or hydrophobic formulation.

33. The cosmetic composition according to claim 31, wherein said cosmetic formulation includes glass flakes in an amount of 0.1-95%, by weight, based on an entirety of said cosmetic formulation.

34. The cosmetic composition according to claim 31, wherein said cosmetic formulation includes a member selected from the group consisting of a polyol, polar or non-polar oil, fat, wax, film former, polymer, copolymer, surfactant, free-radical scavenger, antioxidant, stabilizer, odor enhancer, silicone oil, emulsifier, solvent, preservative, thickener, rheological additive, fragrance, colorant, effect pigments, UV absorber, surface-active assistant and a combination thereof.

35. The cosmetic composition according to claim 31, wherein said cosmetic formulation includes a filler selected from the group consisting of mica, talc, kaolin, sericite, calcium carbonate, silica, BiOCl, barium sulphate, calcium sulphate, boron nitride and a polymer selected from the group consisting of nylon powder, polymethylmethacrylate, a melamine resin and a combination thereof.

36. A method for making glass flakes, comprising the steps of:
feeding molten glass into a cup mounted for rotation about a longitudinal axis;
rotating the cup about said longitudinal axis for causing a film of molten glass to emanate radially from a rim of the cup;
extending an insulating element at least partially around the cup;
subjecting a resultant glass flakes to action of slow fed impact millers under reduced pressure; and,
subjecting the resultant glass flakes to multiple passings through opposed jet mills.

37. A method for making glass flakes, comprising the steps of:
feeding molten glass into a cup mounted for rotation about a longitudinal axis;
rotating the cup about said longitudinal axis for causing a film of molten glass to emanate radially from a rim of the cup;
heating the cup during said rotating step;
subjecting a resultant glass flakes to action of slow fed impact millers under reduced pressure; and,
subjecting the resultant glass flakes to multiple passings through opposed jet mills.
